(19) United States
(12) Patent Application Publication
(10) Pub. No.: US 2008/0044445 A1
(43) Pub. Date: Feb. 21, 2008

(54) Cosmetic Composition and Carrier

(76) Inventor: Patti D. Rubin, Aventura, FL (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/840,086

(22) Filed: Aug. 16, 2007

Related U.S. Application Data
(60) Provisional application No. 60/822,612, filed on Aug. 16, 2006.

(51) Int. Cl. A61K 8/11; A61Q 1/04; A61Q 1/08; A61Q 1/10

(52) U.S. Cl. 424/401; 424/63; 424/64; 424/70.7

(57) ABSTRACT

A cosmetic polymer includes a water absorbent polymer, and a cosmetic ingredient encapsulated in the water absorbent polymer. Encapsulating a cosmetic ingredient in an absorbent polymer allows for systematic release of the cosmetic ingredient, thereby reducing the negative effects of preservatives found in many cosmetic ingredients.

### RELATED APPLICATIONS

The present application claims the benefit under 35 U.S.C. § 119(e) of U.S. Provisional Patent Application No. 60/822,612 filed Aug. 16, 2006 titled “Cosmetic Composition,” which application is hereby incorporated by reference in its entirety.

### BACKGROUND

Cosmetics have been used throughout history to enhance or alter the wearer's appearance. For example, cosmetics are frequently applied to the face to make the wearer more attractive or to disguise blemishes or other imperfections. For many, wearing cosmetics provides the appearance of health and youth. Cosmetics have traditionally been transported in compacts or other small makeup carrying articles. These compacts frequently include either a plurality of different types or a variety of the same type of cosmetic. For example, a compact could carry four different colors of a single eye shadow. Consequently, in order to apply more than one type of cosmetic, multiple compacts are frequently used to hold different types of cosmetic (e.g., one compact holds eye shadow, another compact holds blush, another compact holds powder, etc.). Further, the cosmetics housed by a user in the compacts frequently include preservatives to maintain the cosmetic in the fully constituted state. In particular, preservatives prevent bacteria and fungus from growing in the product and protect products from damage caused by air or light. However, preservatives can also cause the skin to become irritated and infected and are a common cause of skin problems such as clogged pores and acne.

Consequently, a need exists for a space efficient cosmetic transportation and delivery system that allows for multiple types of cosmetics while eliminating or substantially reducing the traditional need for preservatives.

### SUMMARY

According to one exemplary embodiment, a cosmetic delivering polymer system includes a water absorbent polymer and a cosmetic ingredient encapsulated in the water absorbent polymer.

Additionally, according to one exemplary embodiment, an exemplary method for manufacturing a cosmetic includes creating an emulsion of a cosmetic ingredient and a hydrophilic polymer, and encapsulating the cosmetic ingredient in the hydrophilic polymer.

### BRIEF DESCRIPTION OF THE DRAWINGS

The accompanying drawings illustrate various embodiments of the present system and method and are a part of the specification. The illustrated embodiments are merely examples of the present system and method and do not limit the scope thereof.

FIG. 1 is a simple block diagram illustrating a method of forming a cosmetic composition, according to one exemplary embodiment.

FIG. 2 is a simple block diagram depicting a method of forming a cosmetic composition, according to one exemplary embodiment.

FIG. 3 is a simple block diagram illustrating a method of forming a cosmetic composition, according to one exemplary embodiment.

FIG. 4 illustrates a carrier containing a cosmetic composition and its method of use, according to one exemplary embodiment.

Throughout the drawings, identical reference numbers designate similar, but not necessarily identical, elements.

### DETAILED DESCRIPTION

The present specification describes cosmetics that include hydrophilic polymers and at least one cosmetic ingredient or agent encapsulated within the hydrophilic polymer. As used herein, cosmetic shall be broadly understood to mean any composition applied to the epidermis of a person to enhance, cover, or otherwise alter appearance. Furthermore, as used herein and in the appended claims, the combination of the hydrophilic polymer and the cosmetic ingredient may be generally referred to as a “cosmetic polymer.” The resulting cosmetics include at least one cosmetic polymer, and may also include additional ingredients as desired. Additionally, a carrier is provided for containing and transporting the cosmetic polymer. According to one exemplary embodiment, the carrier is in the general shape of a human face. According to such an embodiment, the carrier includes a plurality of compartments located at various locations corresponding to the location of intended use. Particularly, the compartments may correspond to various locations of the face; such as the cheeks, the lips, the eyes, etc. According to one exemplary embodiment, cosmetic polymer is provided in at least one of these locations, which is to be applied to the corresponding area of a user. For example, lip gloss in the form of a cosmetic polymer can be placed in a lip-shaped compartment in the carrier. Further details of the present exemplary system and method for providing cosmetics to a user will be provided below with reference to the figures.

In the following description, for purposes of explanation, numerous specific details are set forth in order to provide a thorough understanding of the present systems and methods. It will be apparent, however, to one skilled in the art that the present apparatus, systems and methods may be practiced without these specific details. Reference in the specification to “an embodiment,” “an example” or similar language means that a particular feature, structure, or characteristic described in connection with the embodiment or example is included in at least that one embodiment, but not necessarily in other embodiments. The various instances of the phrase “in one embodiment” or similar phrases in various places in the specification are not necessarily all referring to the same embodiment.

As mentioned previously, the exemplary cosmetic polymer includes, according to one exemplary embodiment, at least one hydrophilic polymer and at least one cosmetic ingredient or agent encapsulated within the at least one hydrophilic polymer. According to one exemplary embodiment, polymers suitable for use in the cosmetic composition include any suitable polymer including, but not limited to, a wide variety of anionic, cationic, and nonionic materials. Suitable polymers include, but are in no way limited to, acrylic polymers such as acrylamides, acrylates, and co-polymers thereof; poly(alkylene oxides) such as poly(ethylene oxide); cross-linked polyethylene oxide co-polyurethane hydrogel; polyvinyl alcohols; ethylene maleic anhydride copolymer; polyvinylethers; polyacrylic acids; polyvinylpyrrolidones; polyvinylmorpholines; polyamines; polyethyleneimines; polyquaternary ammoniums; saponified copolymers of vinyl acetate-acrylic acid ester; and hydrolyzed copolymers of acrylonitrile or acrylamide, or crosslinked polymers of these hydrolyzed copolymers; partially-neutralized crosslinked poly(acrylic acid); natural based polysaccharide polymers such as methyl celluloses, carboxymethyl celluloses, carboxymethyl starches, hydroxypropyl celluloses, algins, alginates, carrageenans, acrylic grafted starches, acrylic grafted celluloses, chitin, chitosan, starch-acrylonitrile, neutralized graft polymers of starch-acrylic acid; and synthetic polypeptides such as polyaspartic acid, polyglutamic acid, polyasparagins, polyglutamines, polylysines, and polyarginines; as well as the salts, copolymers, cross-linked derivatives and mixtures of any of the foregoing polymers. The polymers may be hydrophilic (water soluble) or hydrophobic (water insoluble) according to various embodiments. Cross-linking hydrophilic polymers can increase the insolubility of the cross-linked compound. Additionally, cross-linked compounds have the capability of absorbing great amounts of water.

In one exemplary embodiment the polymer capsule of the present cosmetic composition is made from a potassium- or sodium-based polymer, such as a synthetic polyacrylate/polyacrylamide copolymer. Like many absorbent polymers, synthetic polyacrylate/polyacrylamide copolymer can absorb many hundred times its weight in water, thereby allowing for a reduction in size of the cosmetic as well as minimizing or reducing the use of preservatives associated with the cosmetic. In another exemplary embodiment, the absorbent polymer capsule is acrylamide/potassium acrylate copolymer.

According to one exemplary embodiment, the polymer capsule carries a cosmetic ingredient that can be released upon hydration to form a fully-hydrated and/or full-sized cosmetic composition that is ready for application. The cosmetic composition may include any number of cosmetic types, such as mascara, eyeliner, eye shadow, foundation, lip gloss, blush, or any other type of cosmetic. Additionally, according to one exemplary embodiment, if the cosmetic polymer is applied with a hydrated applicator, such as a sponge or a brush, only the cosmetic polymers that directly contact the hydrated applicator will be converted to a fully-hydrated and/or full-sized cosmetic composition that is ready for application. The un-hydrated cosmetic polymer will remain in an encapsulated state until activated.

According to one exemplary embodiment, suitable cosmetic ingredients that may be encapsulated by the aforementioned hydrophilic polymers include, but are not limited to acetates, acetones, Acetone, Acetylated Lanolin Alcohol, Acrylates Copolymer, such as Clinac O.C.; Acrylates/Octylpropenamide Copolymer, Alcohol SD-40, Algae/Seaweed Extract, Allantoin, Alpha Hydroxy Acid, such as citric acid, glycolic acid, lactic acid, and malic acid and tartaric acid; Alpha Lipoic Acid, Alum, Ascorbic Acid, Ascorbyl Palmitate, Beeswax, Benzoyl Peroxide, Beta Hydroxy Acid, Boric Acid, Caffeine, Camphor, Carbomers (934, 940, 941, 980, 981) Carmine, Roe Extract, Cellulose, Ceramides, Ceteareth, such as cetearyl and stearyl alcohols, Cetyl Alcohol, Collagen, Cyclic Acid, Cyclomethicone, Dimethicone, EDTA, Elastin, Ellagic Acid, Ethyl Alcohol, glycerin, Glycine, glycogen, Glycolic Acid, Glycol Stearate, Grape Seed Extract, Green Tea Extract, Hyaluronic Acid, Hydroquinone, Isopropyl Alcohol, Isopropyl Isostearate, Isopropyl Palmitate, Isostearic Acid, Kaolin (China Clay), Kojic Acid, Lactic Acid, Lanolin, Lecithin, L-Ergothioneine, Licorice Extract, Linoleic Acid, Lysine Octyl Methoxycinnamate, Octyl Palmitate, Octyl Salicylate, Oxybenzone, Panthenol, Parabens, Poly Hydroxy Acid, Proline, Propylene Glycol, Resveratrol, Retinol, Retinyl Palmitate, Retinyl Palmitate Polypeptide, Rose Hips, Salicylic Acid, Silica, Silicone, Silk Powder, Silk Proteins, Sodium Bicarbonate, Sodium Borate, Sodium Hyaluronate, Sodium Laurel Sulfate, Sorbic Acid, Sorbitol, Stearic Acid, Sulfur, Titanium Dioxide, Triclosan, Tyrosine, Vitamin A, Vitamin B, Vitamin C, Vitamin D, Vitamin E, Witch Hazel, and/or Xanthan Gum. These cosmetic ingredients may be encapsulated or otherwise incorporated as cosmetic polymers or may be included as a cosmetic base. The ingredients included in the cosmetic base may be rehydrated, according to one exemplary embodiment, by the rehydration of the hydrophilic cosmetic polymers.

The concentration of the cosmetic ingredient can be 100% concentrated or alternatively, the cosmetic ingredient may be diluted with water or an organic solvent. According to one exemplary embodiment, the cosmetic ingredient typically ranges from about 0.01% to about 99.9% by weight of the cosmetic polymer. In another embodiment, the cosmetic ingredient ranges from about 0.1% to about 30.0% by weight of the cosmetic polymer. In yet another embodiment the cosmetic agent ranges from about 10% to about 20% by weight of the cosmetic polymer. Essentially any range of cosmetic agent may be included, depending on the capabilities of the polymer.

Generally the cosmetic composition includes one or more polymer capsules in which the cosmetic ingredient inside the capsule is a solid, liquid, or gas. The cosmetic polymers are typically prepared by microencapsulation techniques. Appropriate microencapsulation processes include both physical and chemical techniques, as will be described in further detail below.

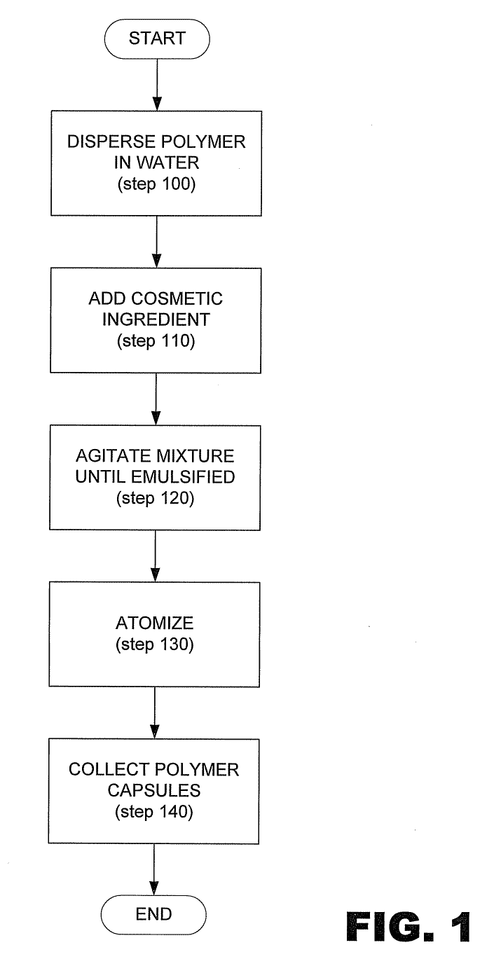

According to one exemplary embodiment, physical methods use commercially available equipment to create and stabilize the capsules housing the cosmetic component. In one exemplary embodiment, the microencapsulation is performed by a spray-drying method, as illustrated in FIG. 1. Generally, the spray drying process begins by creating an emulsion of the capsule polymer and cosmetic ingredient. In one embodiment, as shown in FIG. 1, the emulsion is made by dispersing or dissolving the capsule polymers in a liquid solution such as water (step 100). Once the polymer has been dispersed or dissolved in a solution, the cosmetic ingredient is slowly added (step 110) and the mixture is rapidly agitated until such time as emulsification is complete (step 120).

Once an emulsion has been created, the liquid emulsion is atomized (step 130) into a heated air stream supplied to a drying chamber. The spray-drying process uses a two-nozzle (internal or external mix) assembly, allowing the heated air from an annular geometry to atomize and implode the issuing liquid stream to form fine polymer capsules carrying the microencapsulated cosmetic ingredient in a dispersed state, according to one exemplary embodiment. These atomized particles assume a somewhat spherical shape as they fall through the gaseous medium, and the cosmetic ingredient is encased in the aqueous phase. With high, particle-specific surface areas, heat from the drying chamber flash-evaporates the solvent or aqueous medium, rendering the polymer capsules cyclone-collected into a holding chamber (step 140).

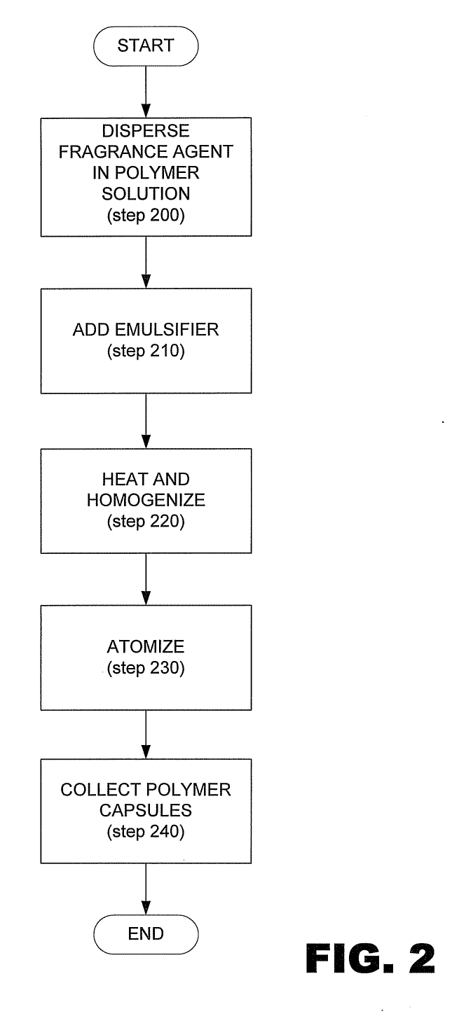

In another embodiment, as shown in FIG. 2, the emulsion is made by dispersing at least one cosmetic ingredient in a polymer solution containing the polymer capsule material to create a dispersion (step 200). An emulsifier is then added to the dispersion (step 210) and the dispersion is then heated and homogenized (step 220). This homogenization creates an oil-in-water type of emulsion. Additionally, a cross-linking agent may be added to the emulsion(s) to enhance the water absorbing ability of the resulting capsule. Once the emulsion has been created, the liquid emulsion is atomized (step 230), as is known in the art, to form polymer capsules. The polymer capsules are then collected (step 240) for use in the cosmetic composition.

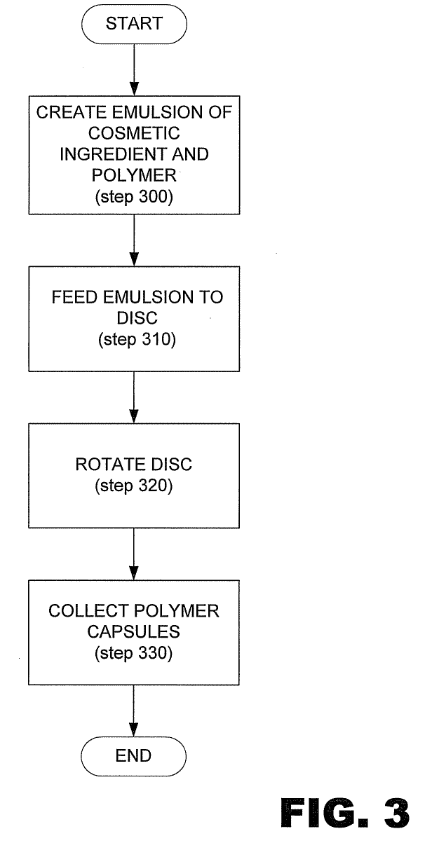

In another exemplary embodiment, illustrated in FIG. 3, the microencapsulation technique used to form the polymer capsules includes a spinning disc process. According to the exemplary method illustrated in FIG. 3, an emulsion or suspension containing the cosmetic ingredient is first prepared (step 300) with a solution or melt of the coating material, similar to the spray-drying process illustrated in FIGS. 1 and 2. Once prepared, the emulsion or suspension is fed to a disc surface (step 310) where it forms a thin wetted layer. Once the emulsion or suspension is fed to the disk surface, the disk is caused to rotate (step 320). During rotation of the disk, the thin layer of emulsion or suspension breaks up into airborne droplets due to surface tension forces that induce thermodynamic instabilities, resulting in spherical capsules that are then collected (step 330). According to one exemplary embodiment, the spinning disk process illustrated in FIG. 3 allows the use of a higher viscosity shell material and allows higher loading of the cosmetic ingredient in the shell. A higher viscosity shell material may be used because the emulsion or suspension is not extruded through an orifice. Additionally, the spinning disk process also offers a broad range of particle sizes that may be formed with controlled distribution by varying the conditions of the rotating disk.

In yet another exemplary embodiment, the microencapsulation technique involves coextrusion encapsulation methods. According to this exemplary embodiment, the polymer capsules may be formed using stationary nozzle coextrusion, centrifugal coextrusion, or submerged nozzle coextrusion. All these processes involve concentric nozzles that pump the cosmetic ingredient through an inner nozzle while the shell formulation is pumped through the annulus, allowing true “core-shell” morphologies. As the liquid stream exits the nozzle, local disturbances, such as induced vibration or gravitational, centrifugal, or drag force, control particle size. Typical microcapsules produced by coextrusion may range from approximately 100 micrometers to 6 mm.

The microencapsulation processes may further include chemical processes such as phase separation, gelation, and simple or complex coacervation. In one exemplary embodiment, the microencapsulation technique involves phase separation in which the cosmetic ingredient is emulsified in a polymer solution and an antisolvent is subsequently added to induce the precipitation of the polymer around the cosmetic ingredient. In another exemplary embodiment coacervation is used. In this technique, microcapsule shells are formed by ionic interaction between two ionic polymers, typically a polyanion and a polycation. In another embodiment, gelation is used as the microencapsulation method and involves using a technique such as cooling, crosslinking, or a chemical reaction to form gelled microspheres or microcapsules.

It should be understood that the present system and methods may also include the use of any other microencapsulation technique known to those of skill in the art. Examples of these methods include, but are not limited to, vibrating nozzle, pan coating, fluid bed, spray coating, interfacial polymerization, solvent evaporation, in situ polymerization, liposome, sol-gel methods, nanoencapsulation, and others.

According to the present exemplary system and method, once the microcapsules are generated, they may then go through any number of processes to prepare the present exemplary cosmetic composition for presentation to a user. According to one exemplary embodiment, the cosmetic composition can be presented to the user in a microencapsulated form. Alternatively, according to one exemplary embodiment, the cosmetic composition may be compressed or otherwise processed such that the cosmetic composition is formed into individual cohesive units, such as thin strips, tabs, capsules, or any other desired units of a desired size. According to one exemplary embodiment, the present exemplary cosmetic composition is formed by joining multiple microcapsules with a hydrophilic binder into a strip form. These strips may then be placed in an appropriate carrier until needed by the user. Such a configuration provides for convenient use of the cosmetic composition by providing a proper amount of the cosmetic composition for a single or multiple applications. Further, such a configuration reduces the volume occupied by the composition, thereby making packing more convenient and further reduces the possibility that the composition will become contaminated.

According to the present exemplary embodiment the polymer encapsulates the cosmetic ingredient, thereby protecting it until the release of the cosmetic ingredient into the environment is induced. Many different mechanisms may trigger the release of the cosmetic ingredient to the environment. In one exemplary embodiment, the polymer is configured to release the cosmetic ingredient through micropores created in the surface of the polymer when the addition of water causes the polymer to swell to a larger size. Release of the cosmetic ingredient in the presence of water may be further facilitated by using hydrophobic cosmetic ingredients that are repelled by absorbed water.

In an alternative exemplary embodiment, the polymer capsule is configured to release the cosmetic ingredient by dissolving in the presence of water. Other release mechanisms that may be used to systematically release the cosmetic ingredient include, but are in no way limited to, mechanical polymer rupture, thermal release, permeation, dissolution, delayed and targeted release, pH and osmotic release, photolytic release, biodegradation, and other release methods known to those of skill in the art.

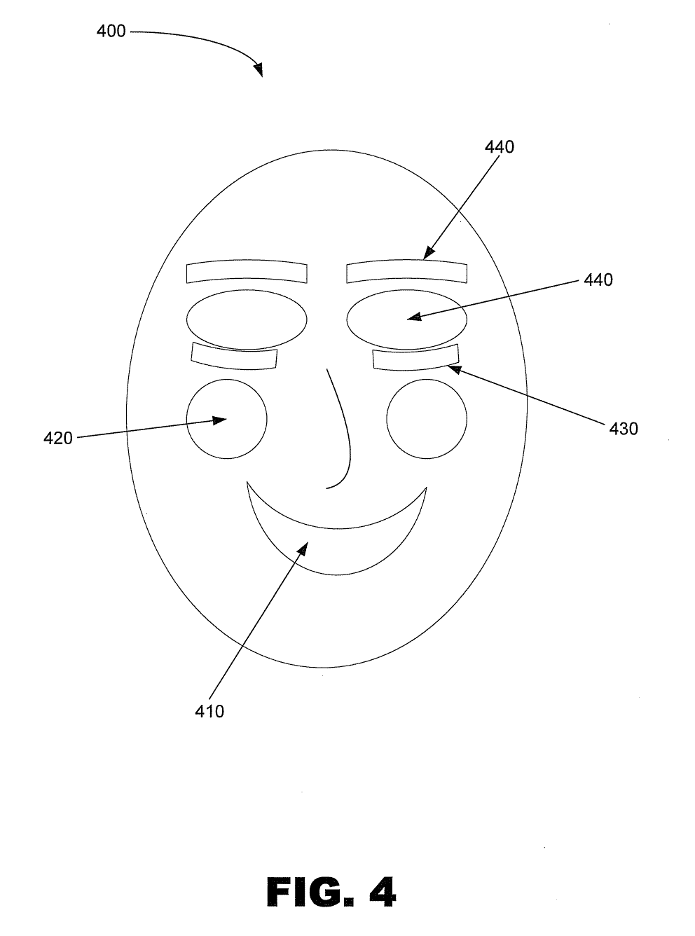

As mentioned, the finished cosmetic polymer composition may be installed into an appropriate carrier such that a user may access the composition when desired. While any number of carriers may be used with the present exemplary composition, FIG. 4 illustrates an exemplary carrier (400) according to one exemplary embodiment. As shown in FIG. 4, the carrier (400) has a general shape and at least minimal features of a human face. Further, according to the present exemplary embodiment, the carrier (400) includes a plurality of compartments located at various locations on the carrier. These compartments may include, according to one exemplary embodiment, a lip gloss compartment (410), blush compartments (420), eye shadow compartments (430), mascara compartments (440), and/or eyebrow cosmetic compartments (450). In this exemplary embodiment, the carrier includes compartments housing cosmetics disposed at locations corresponding to where the cosmetics should be used. This aids not only in identification of the cosmetic being housed in the carrier, but also assures the user that they have an entire spectrum of desired cosmetics.

The preceding description has been presented only to illustrate and describe the present method and products. It is not intended to be exhaustive or to limit the disclosure to any precise form disclosed. Many modifications and variations are possible in light of the above teaching. It is intended that the scope of the present system and method be defined by the following claims.

What is claimed is:

1. A cosmetic composition, comprising:
a water absorbent polymer; and
a cosmetic ingredient encapsulated in said water absorbent polymer.

2. The composition of claim 1, wherein said water absorbent polymer comprises a hydrophilic polymer.

3. The composition of claim 1, wherein said cosmetic ingredient comprises one of a mascara, an eyeliner, an eye shadow, a foundation, a lip gloss, or a blush.

4. The composition of claim 1, wherein said cosmetic ingredient comprises one of acetates, acetones, Acetone, Acetylated Lanolin Alcohol, Acrylate Copolymers, Acrylates/Octylpropenamide Copolymer, Alcohol SD-40, Algae/Seaweed Extract, Allantoin, Alpha Hydroxy Acid, Alpha Lipoic Acid, Alum, Ascorbic Acid, Ascorbyl Palmitate, Beeswax, Benzoyl Peroxide, Beta Hydroxy Acid, Boric Acid, Caffeine, Camphor, Carbomers (934, 940, 941, 980, 981) Carmine, Roe Extract, Cellulose, Ceramides, Ceteareth, Cetyl Alcohol, Collagen, Cyclic Acid, Cyclomethicone, Dimethicone, EDTA, Elastin, Ellagic Acid, Ethyl Alcohol, glycerin, Glycine, glycogen, Glycolic Acid, Glycol Stearate, Grape Seed Extract, Green Tea Extract, Hyaluronic Acid, Hydroquinone, Isopropyl Alcohol, Isopropyl Isostearate, Isopropyl Palmitate, Isostearic Acid, Kaolin (China Clay), Kojic Acid, Lactic Acid, Lanolin, Lecithin, L-Ergothioneine, Licorice Extract, Linoleic Acid, Lysine Octyl Methoxycinnamate, Octyl Palmitate, Octyl Salicylate, Oxybenzone, Panthenol, Parabens, Poly Hydroxy Acid, Proline, Propylene Glycol, Resveratrol, Retinol, Retinyl Palmitate, Retinyl Palmitate Polypeptide, Rose Hips, Salicylic Acid, Silica, Silicone, Silk Powder, Silk Proteins, Sodium Bicarbonate, Sodium Borate, Sodium Hyaluronate, Sodium Laurel Sulfate, Sorbic Acid, Sorbitol, Stearic Acid, Sulfur, Titanium Dioxide, Triclosan, Tyrosine, Vitamin A, Vitamin B, Vitamin C, Vitamin D, Vitamin E, Witch Hazel, or Xanthan Gum.

5. The composition of claim 1, wherein said water absorbent polymer comprises one of an acrylic polymer, a poly alkylene oxide, a cross-linked polyethylene oxide co-polyurethane hydrogel, a polyvinyl alcohol, an ethylene maleic anhydride copolymer, a polyvinylether, a polyacrylic acid, a polyvinylpyrrolidone, a polyvinylmorpholine, a polyamine, a polyethyleneimine, a polyquaternary ammonium, a saponified copolymer of vinyl acetate-acrylic acid ester, a hydrolyzed copolymer of acrylonitrile or acrylamide, a partially-neutralized crosslinked poly(acrylic acid), a natural based polysaccharide polymer, or a synthetic polypeptide.

6. The composition of claim 1, wherein said water absorbent polymer comprises one of a potassium- or sodium-based polymer.

7. The composition of claim 1, wherein said cosmetic ingredient is microencapsulated in said water absorbent polymer.

8. The composition of claim 1, wherein said composition comprises between approximately 0.1% to about 30.0% by weight cosmetic ingredient.

9. The composition of claim 7, wherein said microencapsulated cosmetic ingredient is joined to other microencapsulated hair care ingredients by a hydrophilic binder to form a substrate.

10. The composition of claim 9, wherein said substrate is cut into individual use strips.

11. The composition of claim 9, wherein said microencapsulated cosmetic ingredients further comprise:
a first microencapsulated cosmetic ingredient; and
a second microencapsulated cosmetic ingredient;
wherein said first microencapsulated cosmetic ingredient and said second microencapsulated cosmetic ingredient are mixed prior to being joined by said hydrophilic binder to form said substrate.

12. A cosmetic composition, comprising:
a water activating hydrophilic encapsulation component; and
a cosmetic ingredient encapsulated in said water-absorbent polymer.

13. The composition of claim 12, wherein said water activating hydrophilic encapsulation component is configured to swell when activated.

14. The composition of claim 12, wherein said water activating hydrophilic encapsulation component is configured to dissolve when activated.

15. The composition of claim 12, wherein said cosmetic ingredient comprises one of a mascara, an eyeliner, an eye shadow, a foundation, a lip gloss, or a blush.

16. The composition of claim 12, wherein said water activating hydrophilic encapsulation component comprises one of an acrylic polymer, a poly alkylene oxide, a cross-linked polyethylene oxide co-polyurethane hydrogel, a polyvinyl alcohol, an ethylene maleic anhydride copolymer, a polyvinylether, a polyacrylic acid, a polyvinylpyrrolidone, a polyvinylmorpholine, a polyamine, a polyethyleneimine, a polyquaternary ammonium, a saponified copolymer of vinyl acetate-acrylic acid ester, a hydrolyzed copolymer of acrylonitrile or acrylamide, a partially-neutralized crosslinked poly(acrylic acid), a natural based polysaccharide polymer, or a synthetic polypeptide.

17. A method of producing a cosmetic composition, comprising:
creating an emulsion of a cosmetic ingredient and a hydrophilic polymer; and
encapsulating said cosmetic ingredient in said hydrophilic polymer.

18. The method of claim 17, wherein said step of encapsulating said cosmetic ingredient in said hydrophilic polymer further comprises microencapsulating said cosmetic ingredient in said hydrophilic polymer.

19. The method of claim 18, further comprising binding a plurality of said microencapsulated cosmetic ingredients.

20. The method of claim 19, wherein said binding a plurality of said microencapsulated cosmetic ingredients further comprises creating a substrate of said bound microencapsulated cosmetic ingredients;
cutting said substrate into a plurality of individual portions; and
securing said individual portions to a cosmetic carrier, said cosmetic carrier being shaped as a human face;
wherein a plurality of compartments are defined in said platform, said location of each of said compartments including a cosmetic composition to be applied to a similar location on a user's face.
